(19) United States
(12) Patent Application Publication
(10) Pub. No.: US 2023/0270826 A1
(43) Pub. Date: Aug. 31, 2023

(54) ANTIVIRAL USE OF LIRAGLUTIDE AND GEFITINIB

(71) Applicant: Delta 4 GmbH, Vienna (AT)

(72) Inventor: Paul PERCO, Vienna (AT)

(21) Appl. No.: 18/017,847

(22) PCT Filed: Jul. 30, 2021
(86) PCT No.: PCT/EP2021/071405

§ 371 Date: Jan. 24, 2023

(30) Foreign Application Priority Data
Jul. 30, 2020 (EP) 20188727.0

(51) Int. Cl. A61K 38/26; A61K 31/5377; A61K 31/167; A61K 31/4535; A61K 31/46; A61K 31/473; A61K 38/07; A61K 31/7135; A61K 31/573; A61K 31/445; A61P 31/14

(52) U.S. Cl. CPC A61K 38/26; A61K 31/5377; A61K 31/167; A61K 31/4535; A61K 31/46; A61K 31/473; A61K 38/07; A61K 31/7135; A61K 31/573; A61K 31/445; A61P 31/14

(57) ABSTRACT

The present invention refers to a pharmaceutical preparation comprising liraglutide or gefitinib or a salt, solvate or combination thereof, in an effective amount for use in prophylactic or therapeutic treatment of a disease condition which is caused by or associated with an infection by a coronavirus.

### FIELD OF THE INVENTION

The present invention relates to liraglutide or gefitinib or a salt, or solvate thereof for treating Coronaviridae virus infections.

### BACKGROUND OF THE INVENTION

Coronaviruses are a group of related single-stranded enveloped RNA viruses having a nucleocapsid of helical symmetry. Coronaviruses constitute the subfamily Orthocoronavirinae, in the family Coronaviridae, order Nidovirales, and realm Riboviria.

There are four main virus subgroups (alpha, beta, gamma, and delta-coronavirus) based on their genomic structure.

The genome size of coronaviruses ranges from approximately 26 to 32 kilobases, one of the largest among RNA viruses. They have characteristic club-shaped spikes that project from their surface. Coronaviruses are large, roughly spherical particles with bulbous surface projections. The average diameter of the virus particles is around 125 nm (0.125 μm). The envelope of the virus in electron micrographs appears as a distinct pair of electron-dense shells. The viral envelope consists of a lipid bilayer, in which the membrane (M), envelope (E) and spike (S) structural proteins are anchored. The ratio of E:S:M in the lipid bilayer is approximately 1:20:300. On average a coronavirus particle has 74 surface spikes. A subset of coronaviruses (specifically the members of beta-coronavirus subgroup A) also have a shorter spike-like surface protein called hemagglutinin esterase (HE).

Inside the envelope, there is the nucleocapsid, which is formed from multiple copies of the nucleocapsid (N) protein, which are bound to the positive-sense single-stranded RNA genome in a continuous beads-on-a-string type conformation. The lipid bilayer envelope, membrane proteins, and nucleocapsid protect the virus when it is outside the host cell.

Coronaviruses are susceptible to mutation and recombination and are therefore highly diverse. There are about 40 different varieties and they mainly infect human and non-human mammals and birds. They reside in bats and wild birds, and can spread to other animals and hence to humans.

Until December of 2019, only six different coronaviruses were known to infect humans. Four of these (HCoV-NL63, HCoV-229E, HCoV-OC43 and HKU1) usually caused mild common cold-type symptoms in immunocompetent people and the other two have caused pandemics in the past two decades.

The outbreak of the novel coronavirus SARS-CoV-2, the major culprit for COVID-19 that mainly manifests as respiratory disease, has led to infection of more than 7 million patients in less than 3 months. More than 400,000 patients have died as a consequence of COVID-19, and in many regions the infection rates are still increasing strongly. Currently there is no proven safe and effective therapy available.

Although the body of evidence about the novel coronavirus is rapidly growing, the precise pathobiological mechanisms are still unclear. However due to the previous outbreaks of coronavirus-associated SARS pandemic 2002/2003 (with 8000 infected patients and a mortality of approx. 10%) and the MERS epidemic 2012 (with 2500 infected patients and a mortality of approx. 36%) there is a body of evidence available for closely related viral diseases.

Nakhleh A. et. al. report glycemic control of type 2 diabetic patients with coronavirus disease and possible benefit of insulin therapy (American Journal of Physiology, 2020, 318(6), E835-E837).

Jin T. et. al. report GLP-1 based drugs and possible use thereof for the treatment on COVID-19 disease (Acta Pharmaceutica Sinica B, 2020, 10(7), 1249-1250).

Stoian A. P. et. al. disclose the possible use of DPPIV inhibitors and their role in Covid-19 treatment (Journal of Cardiovascular Pharmacology and Therapeutics, 2020, 25(6), 494-496).

Lee M. Y. and Jin T. report inhibition of Hepatitis C virus replication by liraglutide (International Journal of Molecular Sciences, 2019, 20(18), 4569)

Duparc T. et. al. disclose that liraglutide improves hepatic steatosis and metabolic dysfunctions in an animal model of non-alcoholic steatohepatitis (bioRxiv, preprint, 2019, 1-22).

According to Salgado-Benvindo C. et. al., SARS-CoV-2 infection in cell culture is inhibited by suramin by interfering with early steps of replication cycle. (Antimicrobial Agents and Chemotherapy, 2020, 64(8)).

Smetana K. et. al., disclose raloxifene and bazedoxifene as promising candidates for preventing Covid-19 related cytokine storm and ARDS (International Journal of Experimental and Clinical Pathophysiology and Drug research, 2020, 34(5), 3027-3028).

Shamsi A. et. al. report that glecaprevir and maraviroc are inhibitors of SARS-CoV-2 main protease (Bioscience Reports, 2020, 40(6)).

Lan L. reports the use of maraviroc being an inhibitor of SARS-CoV-2 protease (UK scientists: Zavesca is expected to inhibit the novel coronavirus, 2020, URL:10.1152/ajpendo.00163.2020).

Machado M. E. et. al. report a screening method based on a molecular docking of possible inhibitors of Covid-19 main protease (Microbial Pathogenesis, 2020, 148, 1-6).

Rothan H. A et. al. report that auranofin inhibits coronavirus replication and attenuates inflammation in human cells (Virology, 2020, 547, 7-11).

Gurjar et al. report the screening of anticancer drugs as potential candidates to target COVID-19 disease. (chemRxiv, preprint, 2020, pp. 1-15),

Hondermarck H. et al. discusses the role of growth factor receptors in viral infections. (Faseb BioAdvances, 2020, vol. 2(5), pp. 296-303)

Venkataraman T. and Frieman M. B refer to the role of EGFR signaling in SARS-COV-2 induced pulmonary fibrosis. (Antiviral Research, Elsevier BV, NL, 2017, vol. 143, pp. 142-150).

Opportunities and challenges of repurposing known pharmaceutical compounds are discussed in Schor S. and Einav S. (ACS Infectious Diseases, 2018, vol. 4(2), pp. 88-92).

Taurin S. et al. discuss the use of a combination of gefitinib and raloxifene for the treatment of breast cancer. (Cancer Research, American Association for Cancer Research, US, 2012, vol. 72, Suppl. 8, p. 4669).

CN105138862A reports the use of a combination of gefitinib and quinacrin for the treatment of breast cancer.

Therapeutic options can be divided in vaccines and non-vaccine therapies. Development of safe and effective vaccines for the novel coronavirus will—according to experts—take 18 months in the best case. Non-vaccine therapies can therefore help to slow down spreading of COVID-19 (antiviral drugs) or ameliorate the consequences of respiratory diseases that are often triggered by dysregulation of the immune system and uncontrolled inflammatory reactions (anti-inflammatory drugs).

There is still a high need in antiviral treatments, medicinal and pharmaceutical products which can be used to prevent from virus infection and/or virus spread, in particular in subjects that have been exposed to or infected with a virus, or who are at risk of being infected.

### SUMMARY OF THE INVENTION

The objective is solved by the subject of the present claims and as further described herein.

The invention provides a pharmaceutical preparation comprising liraglutide or gefitinib or a salt, solvate or combination thereof, in an effective amount for use in prophylactic or therapeutic treatment of a disease condition which is caused by or associated with an infection by a coronavirus.

In a specific embodiment, a combination of liraglutide and gefitinib, or the salt or solvate thereof is provided herein for use in prophylactic or therapeutic treatment of a disease condition which is caused by or associated with an infection by a coronavirus.

According to a specific aspect, the coronavirus species are selected from human and non-human, such as zoonotic, Coronaviridae viruses, in particular naturally-occurring coronaviruses including mutants thereof which may evolve during a season of infection or a pandemic, or mutants that are artificially evolved to anticipate naturally-occurring mutants.

Specifically, the coronavirus is a 6-coronavirus, more specifically a human 6-coronavirus. Within the genus 6-coronavirus, four lineages (i.e., A, B, C, and D) are commonly recognized. Specifically, the 6-coronavirus is of the B-lineage (subgenus Sarbecovirus), such as SARS-CoV or SARS-CoV-2, or of the C-lineage (subgenus Merbecovirus), such as MERS-CoV, or of the A-lineage (subgenus Embecovirus), such as HCoV-NL63, HCoV 229E, HCoV-0C43 or HCoV-HKU1.

Specifically, the coronavirus is selected from the group consisting of SARS-CoV-2, MERS-CoV, SARS-CoV-1, HCoV-NL63, HCoV 229E, HCoV-0C43, and HCoV-HKU1, or mutants thereof, in particular those mutants which are naturally-occurring, i.e which are not artificial (“man-made”) but found in nature.

According to a specific aspect, the pharmaceutical preparation is a medicinal product or a drug product, comprising liraglutide or gefitinib and a pharmaceutically acceptable carrier.

Specifically, the pharmaceutical preparation described herein is used for treatment or prevention of a disease condition which can be, but is not limited to common cold, infection of the nose, throat and larynx, sinusitis, bronchiolitis, diarrhea, rash on skin, pneumonia, or acute respiratory distress syndrome (ARDS), symptoms of the central nervous system, hepatic steatosis, portal fibrosis, occurrence of lymphocytic infiltrates and ductular proliferation, lobular cholestasis, acute liver cell necrosis, central vein thrombosis, renal proximal tubular injury, focal pancreatitis, adrenocortical hyperplasia, and lymphocyte depletion of spleen and lymph nodes, alveolar damage specifically characterized by edema, hyaline membranes, and proliferation of pneumocytes and fibroblasts, endothelial damage.

Specifically, the effective amount is effective in preventing infection of susceptible cells by the virus, thereby treating the disease condition.

Specifically, the effective amount is effective by reducing or inhibiting inflammatory and undesired coagulation symptoms associated with virus infection.

In a specific embodiment, the effective amount is administered locally or systemically.

Liraglutide is a synthetic analog of human glucagon-like peptide-1(GLP-1) and acts as a GLP-1 receptor agonist. Liraglutide is 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. Liraglutide comprises the sequence:

His-Ala-Glu-Gly-Thr-Phe-Thr-Ser-Asp-Val-Ser-Ser-Tyr-Leu-Glu-Gly-Gln-Ala-Ala-Lys(1)-Glu-Phe-Ile-Ala-Trp-Leu-Val-Arg-Gly-Arg-Gly (SEQ ID NO: 1).

Its protein chemical formula is C₁₇₂H₂₆₅N₄₃O₅₁.

Liraglutide is commercially available under the trade names Saxenda® and Victoza®.

Liraglutide has cardio-, reno-, as well as neuroprotective effects. Liraglutide was also shown to counterbalance inflammatory processes and oxidative stress. Levels of TNF, IL1b, IL6, IL17 and IL21, key players in inflammatory response after COVID-19 infection, are reduced by liraglutide. In addition, liraglutide modulates DPP4 levels, a molecule potentially linked to coronavirus entry and replication.

Specifically, TNF, IL17, IL21, IL6 and/or IL1b are inhibited by liraglutide.

In a further embodiment, gefitinib is present in the pharmaceutical preparation described herein. Gefitinib is characterized by the structure (I)

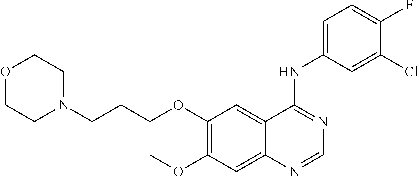 [SMILES: COc1cc2ncnc(Nc3ccc(F)c(Cl)c3)c2cc1OCCCN1CCOCC1]

or is a pharmaceutically acceptable salt or solvate thereof.

Its protein chemical formula is C₂₂H₂₄ClFN₄O₃
Gefitinib is commercially available under the trade name Iressa®.
More specifically, EGFR signaling pathway is inhibited by gefitinib.

There is evidence that EGFR inhibitors modulate expression of CEACAM1 and CD9, two molecules being reported to play a role in viral entry and replication. Inhibition of the EGFR signaling pathway itself may lead to amelioration of pulmonary fibrosis after CoV infection. Reduction of FGF2 by EGFR inhibition in addition might lower the rate of apoptosis in renal and lung cells thus alleviating COVID-19 severity. The link of EGFR inhibition to inflammation is ambivalent with evidence available that EGFR inhibition reduces TNF but reports are also showing that TNF levels are enhanced after EGFR inhibition. A number of molecules associated with SARS CoV-2 infection are in addition capable of modulating resistance to EGFR inhibition based on a number of studies from the field of oncology where EGFR inhibition is primarily investigated.

Specifically, the pharmaceutical preparation is formulated for systemic administration, preferably by intravenous, intramuscular, subcutaneous, intradermal, transdermal, or oral administration or for local administration, specifically for application to the upper and lower respiratory tract, nasal, pulmonary, intraoral, ocular, or dermal use.

Specifically, a liquid solution or dispersion containing liraglutide is used for parenteral administration, such as by inhalation, infusion or injection, preferably wherein the effective amount provides about 50 ng to 1 g per dose. Specifically, liraglutide is administered by subcutaneous injection.

Specifically, the preparation comprises about 50 ng to 1 g/dose of liraglutide, more specifically the preparation comprises about 100 ng to 800 mg, specifically 500 ng to 500 mg/dose.

Specifically, the daily dose of liraglutide is in the range of about 50 ng to 1 g of liraglutide, more specifically the preparation comprises about 100 ng to 800 mg, specifically 500 ng to 500 mg/dose.

Specifically, gefitinib is formulated as tablet or capsule or nano-capsule or liposomal preparation for oral administration, preferably wherein the effective amount provides about 50 ng to 1 g per dose. Specifically, a tablet comprising gefitinib may be used which can be administered once to several times a day, specifically once to three times a day.

Specifically, the preparation comprises about 50 ng to 1 g/dose of gefitinib, more specifically the preparation comprises about 100 ng to 800 mg, specifically 500 ng to 500 mg/dose.

Specifically, the daily dose is in the range of about 50 ng to 1 g of gefitinib, more specifically the preparation comprises about 100 ng to 800 mg, specifically 500 ng to 500 mg.

According to an embodiment, the pharmaceutical preparation is administered to the subject as a spray, a powder, a gel, an ointment, a cream, a foam, or a liquid solution, a lotion, a patch, a gargle solution, an aerosolized powder, an aerosolized liquid formulation, granules, capsules, specifically comprising nanoparticles, drops, tablet, syrup, lozenge, nano-capsule, liposomal preparation or a preparation for infusion or injection.

According to a specific embodiment, liraglutide can be administered by inhalation, more specifically in a dose of about 1 to 15 μg/kg, specifically 2, 3, 4, 5, 6, 7, 8, 9, 10, 11, 12 13, 14, 15 μg/kg.

Specifically, the preparation is administered as the sole substance, or wherein treatment is combined with a further treatment with one or more active substances, preferably selected from the group consisting of antiviral, anti-inflammatory and antibiotic substances.

In a specific embodiment, the preparation is administered in combination with an active agent selected from the group consisting of suramin, raloxifene, maraviroc, miglustat, quinacrine, glatiramer acetate, auranofin and dexamethasone.

Specifically, a subject is treated with the preparation described herein, who has been infected or is at risk of being infected with coronavirus.

According to a specific aspect, a subject is treated who has been infected or is at risk of being infected with said virus, preferably a human being, or a non-human mammal, such as a dog, cat, horse, camelids, cattle or pig.

Specifically, the subject is or has been exposed to a virus, or is otherwise at risk of being infected with the virus.

Specifically, the subject has been determined or diagnosed of being infected with the virus.

In specific embodiments, a subject is treated which is a diseased subject or patient suffering from Coronaviridae virus-caused disease, such as gastroenteritis, respiratory tract disease, or severe acute respiratory syndrome (SARS). Specifically, the disease is a β-coronavirus-caused disease e.g., a SARS virus-caused disease, upon getting in contact with the pathogen, such as COVID19, or COVID19-associated pneumonia.

According to a further embodiment, there is provided a pharmaceutical preparation comprising liraglutide or gefitinib or a salt, solvate or combination of liraglutide and gefitinib, in an effective amount, further comprising an active agent selected from the group consisting of suramin, raloxifene, maraviroc, miglustat, quinacrine, glatiramer acetate and auranofin.

According to a specific aspect, a kit is provided which comprising one or more individual dosage units of liraglutide, or gefitinib as further described herein, and directions for their use in treating a coronavirus infection or a coronavirus-caused disease, in a human or non-human mammal.

According to a specific aspect, the invention further provides for methods of treating a subject being infected or at risk of being infected with a virus such as a coronavirus, comprising administering an effective amount of liraglutide or gefitinib or combination thereof, and respective medicinal products or pharmaceutical preparations as further described herein.

According to a further specific aspect, the invention provides for an preparation of liraglutide or gefitinib as described herein (such as a medicinal product, pharmaceutical preparation or disinfectant) and methods of producing such antiviral preparation comprising formulating an antiviral effective amount of liraglutide, or gefitinib, with a pharmaceutically acceptable carrier to produce a preparation, in particular a medicinal product or pharmaceutical preparation which specifically has antiviral, anti-inflammatory and/or anti-coagulation effect.

### FIGURES

FIG. 1: Cytokine release assay of interleukin 6 (IL-6) in response to nucleocapsid (N) stimulation or Mock stimulation as control. Immune modulation by gefitinib (GEF) alone was significantly stronger than by dexamethasone (DEX) alone. Numbers at the bottom of the boxplot represent replicate numbers of the experiments. The strongest immunomodulatory effect was observed in combination of GEF and DEX.

FIG. 2: Cytokine release assay of interleukin 17 (IL-17), tumor necrosis factor beta (TNF-beta) and IL-21 in response to nucleocapsid (N) stimulation or Mock stimulation as control. Immune modulation by gefitinib (GEF, 5 μM), liraglutide (LIR, 2 μg/ml), and dexamethasone (DEX, 5 μM) alone as well as in combination is shown relative to stimulation with the positive control resiquimod (R848, 200 ng/ml).

FIG. 3: Cytokine release assay of interleukin 6 (IL-6) in response to spike (S), spike trimer (St) or Mock stimulation as control. Immune modulation by gefitinib (GEF, 0.5 μM and 5 μM) alone is shown in comparison to pomalidomide (POM, 100 ng/ml) which does not show immune modulation. Numbers at the bottom of the bar chart represent replicate numbers of the experiments.

FIG. 4: Genes from the COVID-19 model are displayed that were up- or down-regulated 2-fold in the comparison of SARS-CoV2 nucleocapsid (i.e. upon stimulation with nucleocapsid (N) protein) vs Mock are “normalized” in their expression by both gefitinib (GEF) and liraglutide (LIR).

### DETAILED DESCRIPTION

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

Compounds such as gefitinib and liraglutide as described herein may be used as a “physiologically acceptable salt”. The choice of salt is determined primarily by how acid or basic the chemical is (the pH), the safety of the ionized form, the intended use of the drug, how the drug is given (for example, by mouth, injection, or on the skin), and the type of dosage form (such as tablet, capsule, or liquid).

Exemplary salts which are physiologically acceptable are sodium salts. However, it is also possible to employ, in place of the sodium salts, other physiologically acceptable salts, e.g., other alkali metal salts, alkaline earth metal salts, ammonium salts and substituted ammonium salts. Specific examples are potassium, lithium, calcium, aluminum and iron salts. Preferred substituted ammonium salts are those derived, for example, from lower mono-, di-, or trialkylamines, or mono-, di- and trialkanolamines. The free amino acids per se can also be used. Specific examples are ethylamine, ethylenediamine, diethylamine, or triethylamine salts.

The term “pharmaceutically acceptable” also referred to as “pharmacologically acceptable” means compatible with the treatment of animals, in particular, humans. The term pharmacologically acceptable salt also includes both pharmacologically acceptable acid addition salts and pharmacologically acceptable basic addition salts.

The term “pharmacologically acceptable acid addition salt” as used herein means any non-toxic organic or inorganic salt of any base compound of the disclosure, or any of its intermediates. Basic compounds of the disclosure that may form an acid addition salt include, for example, compounds that contain a basic nitrogen atom. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono-, di- or the triacid salts can be formed, and such salts may exist in either a hydrated, solvated or substantially anhydrous form. In general, the acid addition salts of the compounds of the disclosure are more soluble in water and various hydrophilic organic solvents, and generally demonstrate higher melting points in comparison to their free base forms. The selection of the appropriate salt will be known to one skilled in the art. Other non-pharmacologically acceptable acid addition salts, e.g. oxalates, may be used, for example, in the isolation of the compounds of the disclosure, for laboratory use, or for subsequent conversion to a pharmacologically acceptable acid addition salt. Specifically, gefitinib may be present as gefitinib hydrochloride salt.

Liraglutide may also be present in the composition in the form of base or in the form of its salts or mixtures thereof. Representative example of salts includes salts with suitable inorganic acids such as hydrochloric, hydrobromic, and the like. Representative examples of salts also include salts with organic acids such as formic acid, acetic acid, propionic acid, lactic acid, tartaric acid, ascorbic acid and the like. Representative examples of salts also include salt with base such as triethanolamine, diethylamine, meglumine, arginine, alanine, leucine, diethylethanolamine, olamine, triethylamine, tromethamine, choline, trimethylamine, taurine, benzamine, methylamine, dimethylamine, trimethylamine, methylethanolamine, propylamine, isopropylamine, adenine, guanine, cytosine, thymine, uracil, thymine, xanthine, hypoxanthine and like. Liraglutide may also be present as liraglutide acetate. In another embodiment, liraglutide is present as a tromethamine salt.

Liraglutide may also be present as functional variant or conjugate.

The term “solvate” refers to a compound in the solid state, where molecules of a suitable solvent are incorporated in the crystal lattice. A suitable solvent for therapeutic administration is physiologically tolerable at the dosage administered. Examples of suitable solvents for therapeutic administration are ethanol and water, but may also be isopropanol, methanol, dimethyl sulfoxide, ethyl acetate, acetic acid and amino ethanol. When water is the solvent, the solvate is referred to as a hydrate. In general, solvates are formed by dissolving the compound in the appropriate solvent and isolating the solvate by cooling or using an antisolvent. The solvate can be dried or azeotroped under ambient conditions.

The term “effective amount” with respect to an antiviral, anti-inflammatory or anti-coagulant effect as used herein, shall refer to an amount (in particular a predetermined amount) that has a proven antiviral, anti-inflammatory or anti-coagulant effect. The amount is typically a quantity or activity sufficient to, when administered to a subject effect beneficial of desired results, including antiviral or clinical results, and, as such, an effective amount or synonym thereof depends upon the context in which it is being applied.

An effective amount of a pharmaceutical preparation or drug is intended to mean that amount of a compound that is sufficient to treat, prevent or inhibit a disease, disease condition or disorder. Such an effective dose specifically refers to that amount of the compound sufficient to result in healing, prevention or amelioration of conditions related to diseases or disorders described herein.

In the context of disease, effective amounts (in particular prophylactically or therapeutically effective amounts) of liraglutide, or gefitinib as described herein are specifically used to treat, modulate, attenuate, reverse, or affect a disease or condition that benefits from its antiviral, anti-inflammatory or anti-coagulation effect. The amount of the compound that will correspond to such an effective amount will vary depending on various factors, such as the given drug or compound, the formulation, the route of administration, the type of disease or disorder, the identity of the subject or host being treated, the assessment of the medical situations and other relevant factors, but can nevertheless be routinely determined by one skilled in the art.

The term “antiviral” as used herein shall refer to any substance, drug or preparation, that effects the biology of a virus and attenuates or inhibits viral attachment, entry, replication, shedding, latency or a combination thereof, resulting in reduction of viral load or infectivity. The terms “attenuating”, “inhibiting”, “reducing”, or “preventing”, or any variation of these terms, when used in the claims and/or the specification includes any measurable decrease or complete inhibition to achieve a desired result, e.g., reduction in the risk of viral infection (pre-exposure), or reduction of post-exposure viral survival, load, or growth.

The term “anti-coagulant” as used herein shall refer to any substance, drug or preparation, that effects coagulation of blood. The terms “attenuating”, “inhibiting”, “reducing”, or “preventing”, or any variation of these terms, when used in the claims and/or the specification includes any measurable decrease or complete inhibition to achieve a desired result, e.g., prolonging clotting time.

The term “anti-inflammatory” refers to any substance, drug or preparation, that reduces inflammation or swelling.

A treatment or prevention regime of a subject with an effective amount of liraglutide, gefitinib or combination thereof described herein may consist of a single application or administration, or alternatively comprise a series of applications and administrations, respectively. For example, gefitinib, or liraglutide may be used at least once a month, or at least once a week, or at least once a day. However, in certain cases of an acute phase, e.g. upon suspected or confirmed exposure to a virus, or after virus infection has been determined, gefitinib, or liraglutide may be used more frequently, e.g. 1-10 times a day.

Specifically, a combination therapy is provided which includes treatment with the preparation described herein and standard therapy of a coronavirus-caused disease.

Doses may be applied in combination with other active agents such as antiviral agents, anti-inflammatory drugs or antibiotics, e.g. upon the subject's risk of viral spread, so to prevent a pathogen associated reaction.

Treatment can be combined with an antiviral, anti-inflammatory or antibiotic treatment, preferably wherein a pharmaceutical preparation is administered before, during (e.g., by co-administration or in parallel), or after said antiviral, anti-inflammatory or antibiotic treatment. The agents can be in separate containers or mixed in a single container.

The length of the treatment period depends on a variety of factors, such as the severity of the disease, either acute or chronic disease, the age of the patient, and the concentration of gefitinib, liraglutide or combination thereof. It will also be appreciated that the effective dosage used for the treatment or prophylaxis may increase or decrease over the course of a particular treatment or prophylaxis regime. Changes in dosage may result and become apparent by standard diagnostic assays known in the art.

Specifically, the preparation is administered in combination with one or more of suramin, raloxifene, maraviroc, miglustat, quinacrine, glatiramer acetate and auranofin.

Specifically, the preparation comprises one or more of suramin, raloxifene, maraviroc, miglustat, quinacrine, glatiramer acetate, auranofin and dexamethasone.

Suramin is an anti-parasitic gent. Suramin has the molecular formula C₅₁H₄₀N₆O₂₃S₆ and is of following structure:

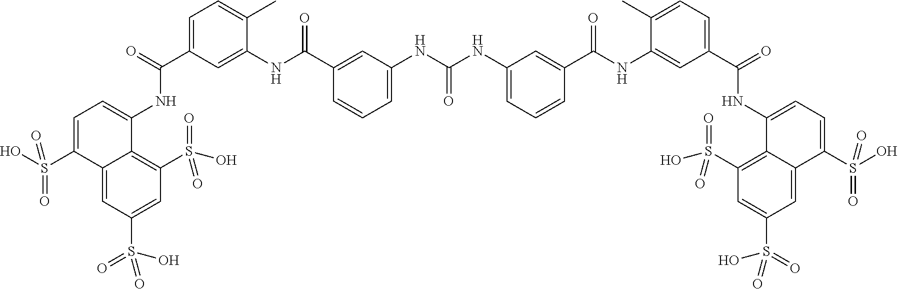 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

Raloxifene (Evista™) is an estrogen receptor modulator and has the structure:

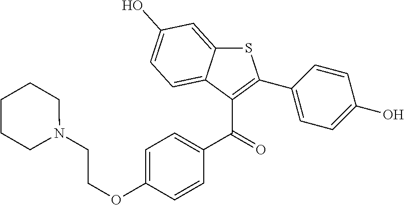 [SMILES: O=C(c1ccc(OCCN2CCCCC2)cc1)c1c(-c2ccc(O)cc2)sc2cc(O)ccc12]

Maraviroc, a 4,4-Difluoro-N—R1S)-3-{(1R,3s,5S)-3-[3-methyl-5-(propan-2-yl)-4H-1,2,4-triazol-4-yl]-8-azabicyclo[3.2.1]octan-8-yl}-1-phenylpropyl] cyclohexanecarboxamide is an anti-infective agent and CCR5 co-receptor antagonist. It has the structure

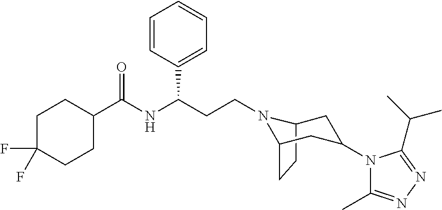 [SMILES: Cc1nnc(C(C)C)n1C1CC2CCC(C1)N2CC[C@H](NC(=O)C1CCC(F)(F)CC1)c1ccccc1]

Miglustat (N-butyl-deoxynojirimycin, N-butylmoranoline), is a small molecule effective as anti-infective agent having the following structure

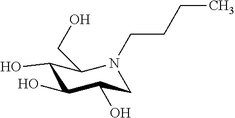

Quinacrine (Mepacrine) is a medication with several uses. It is related to chloroquine and mefloquine, and has the structure

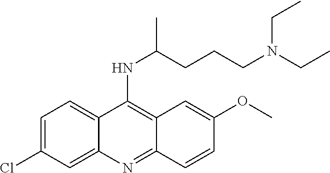 [SMILES: CCN(CC)CCCC(C)Nc1c2ccc(Cl)cc2nc2ccc(OC)cc12]

Glatiramer acetate (also known as Copolymer 1, Cop-1, or Copaxone) is an immunomodulator medication currently used to treat multiple sclerosis. It has the structure

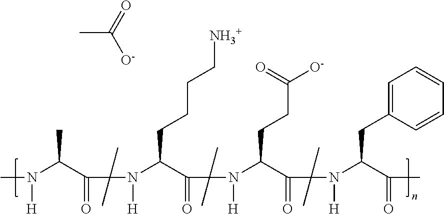

Auranofin (brand name Ridaura™) is an anti-inflammatory agent and has the structure

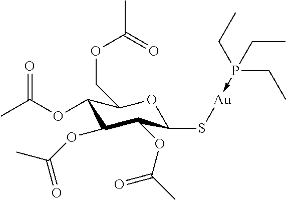

Dexamethasone is a glucocorticoid medication, used in the treatment of inflammatory and autoimmune diseases and is sold under the brand names Dextenza®, Ozurdex®, Neofordex®. It has the structure

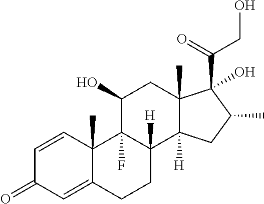

Dexamethasone may be administered in the form of a tablet, specifically containing the compound in an amount of about 0.1 to 10 mg/tablet, specifically about 0.5, 0.75, 1, 1.5, 2, 3, 4, 5, 6, 7, 8, 9, 10 mg.

Dexamethasone may be administered as solution, specifically in a range of 0.1 to 5 mg/dose, specifically 0.1, 0.2, 03, 0.4, 0.5, 0.6, 0.7, 0.7, 0.9, 1, 2, 3, 5, 5 mg/dose.

The preparation described herein may be provided for single or multiple dosage use.

Unit-dose or multi-dose containers may be used, for example, sealed ampoules and vials, or multi-use sprays, and may be stored comprising a liquid or dry phase, e.g., in a freeze-dried (lyophilized) condition requiring only the addition of the sterile liquid carrier, for example water for injection, immediately prior to use. Preferred unit dosage formulations are those containing a daily dose or unit daily sub-dose, or multiple doses, comprising liraglutide or gefitinib or a salt, solvate or combination thereof.

A single-dose or amount for single-use is the amount intended for administration that is meant for use in a single subject, such as a patient, either human or animal for a single case/procedure/administration. Packages comprising the single-dose are typically labelled as such by the manufacturer. The single-dose amount is specifically understood as a daily dose for an individual, like a child or adult, to provide an effective amount.

The pharmaceutical preparation or medicinal product described herein is specifically provided as human or veterinary pharmaceutical composition or medicinal product. Medicinal products are understood as substances that are used to treat diseases, to relieve complaints, or to prevent such diseases or complaints in the first place. This definition applies regardless of whether the medicinal product is administered to humans or to animals. The substances can act both within or on the body.

The pharmaceutical preparation described herein preferably contains one or more pharmaceutically acceptable auxiliaries and is in a pharmaceutical form which allows the active pharmaceutical compound to be administered with high bioavailability. Suitable auxiliaries may be, for example, based on cyclodextrins. Suitable formulations might for example incorporate synthetic polymeric nanoparticles formed of a polymer selected from the group consisting of acrylates, methacrylates, cyanoacrylates, acrylamides, polylactates, polyglycolates, polyanhydrates, polyorthoesters, gelatin, albumin, polystyrenes, polyvinyls, polyacrolein, polyglutaraldehyde and derivatives, copolymers and mixtures thereof.

Specific medicinal products or pharmaceutical compositions described herein comprise liraglutide or gefitinib or combination thereof and a pharmaceutically acceptable carrier or excipient.

A “pharmaceutically acceptable carrier” refers to an ingredient in a formulation for medicinal or medical use, other than an active ingredient, which is nontoxic to a subject. A pharmaceutically acceptable carrier includes, but is not limited to, a buffer, excipient, stabilizer, or preservative, and the like.

Liraglutide or gefitinib as used herein can be formulated with conventional carriers and excipients, which will be selected according ordinary practice.

Commercially available liraglutide and gefitinib formulations may also be used for the prophylactic or therapeutic treatment of a disease condition which is caused by or associated with an infection by a coronavirus described herein.

Pharmaceutically acceptable carriers generally include any and all suitable solvents, dispersion media, coatings, antiviral, antibacterial and antifungal agents, isotonic and absorption delaying agents, and the like that are physiologically compatible with an antiviral small molecule compound or related composition or combination preparation described herein.

According to a specific aspect, liraglutide, gefitinib or a salt, solvate or combination thereof can be combined with one or more carriers appropriate a desired route of administration. Liraglutide, gefitinib or combination thereof may be e.g., admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidone, polyvinyl alcohol, and optionally further tableted or encapsulated for conventional administration. Alternatively, liraglutide and gefitinib may be dispersed or dissolved in saline, water, polyethylene glycol, propylene glycol, carboxymethyl cellulose colloidal solutions, ethanol, corn oil, peanut oil, cotton seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well-known in the art.

The compounds as described herein may be provided in controlled release pharmaceutical (“controlled release formulations”) in which the release of liraglutide or gefitinib is controlled and regulated to allow less frequency dosing or to improve the pharmacokinetic or toxicity profile of a given active ingredient.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Additional pharmaceutically acceptable carriers are known in the art and described in, e.g., Remington: The Science and Practice of Pharmacy, 22^{nd} revised edition (Allen Jr, LV, ed., Pharmaceutical Press, 2012). Liquid formulations can be solutions, emulsions or suspensions and can include excipients such as suspending agents, solubilizers, surfactants, preservatives, and chelating agents.

The preferred preparation is in a ready-to-use, storage stable form, with a shelf-life of at least one or two years.

The term “formulation” as used herein refers to a preparation ready-to-use in a specific way. Specifically, compositions described herein comprises liraglutide or gefitinib or a salt, solvate or combination thereof, and a pharmaceutically acceptable diluent, carrier or excipient.

Specifically, gefitinib can be orally administered, for example, with an inert diluent or an assimilable or edible carrier. For example, a preparation may be enclosed in a hard- or soft-shell gelatin capsule, or compressed into tablets. For oral therapeutic administration, gefitinib may be incorporated with excipients and used in the form of ingestible tablets, buccal tablets, troches, capsules, elixirs, suspensions, syrups, wafers, and the like. The percentage of the compound in the compositions and preparations may, of course, be varied. The amount of gefitinib in therapeutically useful compositions is such that a suitable dosage will be obtained.

Tablets will contain excipients, glidants, fillers, binders, disintegrants, lubricants, flavors and the like. Granules may be produced using isomaltose. It is furthermore preferred to provide for a preparation formulated to act at the site of the mucosa, e.g. at mucosal sites (such as nose, mouth, eyes, esophagus, throat, lung), e.g. locally without systemic action. Aqueous formulations are prepared in sterile form, and when intended for delivery by other than oral administration generally will be isotonic.

Pharmaceutical compositions suitable for injectable use, specifically for administering liraglutide, include sterile aqueous solutions (in particular where the compounds or pharmaceutically acceptable salts are water soluble) or dispersions and sterile powders for the extemporaneous preparation of sterile injectable solutions or dispersion. In particular, the composition is specifically sterile and fluid to the extent that easy syringability exists; it is stable under the conditions of manufacture and storage and preserved against the contaminating action of microorganisms such as bacteria and fungi.

Suitable pharmaceutically acceptable vehicles include, without limitation, any non-immunogenic pharmaceutical adjuvants suitable for oral, parenteral, nasal, mucosal, transdermal, intravascular, intraarterial, intramuscular, and subcutaneous administration routes, such as phosphate buffer saline.

Liraglutide and gefitinib may be administered simultaneously or successively.

The term “subject” as used herein shall refer to a warm-blooded mammalian, particularly a human being or a non-human animal, including e.g., dogs, cats, rabbits, horses, cattle, and pigs. In particular the treatment and medical use described herein applies to a subject in need of prophylaxis or therapy of a disease condition associated with a coronavirus infection. Specifically, the treatment may be by interfering with the pathogenesis of a disease condition where a coronavirus is a causal agent of the condition. The subject may be a patient at risk of such disease condition or suffering from disease.

The term “at risk of” a certain disease conditions, refers to a subject that potentially develops such a disease condition, e.g., by a certain predisposition, exposure to virus or virus-infected subjects, or that already suffers from such a disease condition at various stages, particularly associated with other causative disease conditions or else conditions or complications following as a consequence of viral infection. The risk determination is particularly important in a subject, where a disease has not yet been diagnosed. This risk determination therefore includes early diagnosis to enable prophylactic therapy. Specifically, liraglutide or gefitinib or a salt, solvate or combination thereof is used in subjects with a high risk, e.g. a high probability of developing disease.

The term “patient” includes human and other mammalian subjects that receive either prophylactic or therapeutic treatment. The term “patient” as used herein always includes healthy subjects. The term “treatment” is thus meant to include both prophylactic and therapeutic treatment.

Specifically, the term “prophylaxis” refers to preventive measures which is intended to encompass prevention of the onset of pathogenesis or prophylactic measures to reduce the risk of pathogenesis.

The term “therapy” as used herein with respect to treating subjects refers to medical management of a subject with the intent to cure, ameliorate, stabilize, reduce the incidence or prevent a disease, pathological condition, or disorder, which individually or together are understood as “disease condition”. The term includes active treatment, directed specifically toward the improvement of a disease condition, prophylaxis directed specifically toward the prevention of a disease condition, and also includes causal treatment directed toward removal of the cause of the associated disease condition. In addition, this term includes palliative treatment designed for the relief of symptoms rather than the curing of the disease condition, and further curing a disease condition directed to minimizing or partially or completely inhibiting the development of the associated disease condition, and supportive treatment employed to supplement another specific therapy directed toward the improvement of the associated disease condition.

The foregoing description will be more fully understood with reference to the following examples. Such examples are, however, merely representative of methods of practicing one or more embodiments of the present invention and should not be read as limiting the scope of invention.

### EXAMPLES

### Example 1

In Vitro Testing

Peritoneal immune cells as well as peripheral blood mononuclear cells (PBMC) provide an optimal in vitro test system to copy, as a surrogate system, the sensor functions of the innate immunity against infectious agents and, in the case of PBMC, to provide the link to adaptive immunity in cell culture. The clearance and control of viruses in the body depends on the cascade-like orchestrated functions of the innate and adaptive immune system and its efficiency in overcoming viral infection. The innate immune response (Innate immunity/first line of defense mechanisms) to the majority of viral infections essentially comprises NK (Natural Killer) cells and type I interferons (IFN) as key regulators. In addition to Type I IFNs, which are mainly produced by plasmocytoid dendritic cells (pDCs), high levels of IL12 and IL18 produced by conventional DCs may play a role. Besides T cells and mast cells, other myeloid cell types such as macrophages or neutrophil granulocytes contribute to orchestrating the early response to viral infections. The composition of immune cells in the PD effluate represents a representative population for the representation of the Innate Immunity, and allows a read-out in a highly-abundant biological waste material (typically 90% monocytes/macrophages, 5% lymphocytes, 3% neutrophil granulocytes, 2% others). In several preliminary studies this circumstance was exploited to develop a clinically applicable assay of ex-vivo stimulated cytokine release for testing peritoneal immune competence (Herzog et al. Sci Rep 2017, PMID: 28740213) and successfully used as primary parameter of an interventional randomized controlled phase II study in patients undergoing peritoneal dialysis (PD) (Vychytil et al. Kidney Int 2018, 94(6) 1227-1237, PMID: 30360960). Alternative stimulants (e.g. viral antigens, live virus or pseudo-viral substances such as poly:IC) can also be used in the present assay. This assay has been adapted for viral infections (stimulation conditions, read-out) and thus represents a novel tool. In combination, these well-established in vitro test systems form a read-out system that has proven to be a valuable tool for the analysis of immunological activity profiles against bacterial and viral agents both for the innate immunity and for the interface to the adaptive immunity (Sadeghi et al. J Infect Dis 2007 PMID:17191175; Sadeghi et al PLoS ONE 2016, PMID:27695085; Wisgrill et al J Leukoc Biol 2016 PMID:26965638; Wisgrill et al J Leukoc Biol 2019 PMID:31211458). This immune-profiling system is applied to a SARS-CoV-2 infection model.

Culture and Stimulation of Cells

PBMCs/ml (1×10⁶) were cultured in AIM-V cell culture medium (Thermo Fisher Scientific), supplemented with IL-3 (10 ng/ml; PeproTech, Rocky Hill, N.J., USA). Cells were stimulated with SARS-CoV2 proteins (spike or nucleocapsid) or left untreated (mock) for 4-6 or, 20-22 hrs. Cultures were incubated in a humidified 5% CO₂ environment at 37° C. For intracellular cytokine experiments, 1.5 M monensin was added additionally after 2 h to all wells to block intracellular protein transport. After 8, 20, or 40 h, cells were harvested on ice, and supernatants were frozen at −80° C. for further cytokine analysis with ELISA (Schüller S. et al. (J. Leukocyte Biol., 93, 2013, 781-787).

Cytokine Release Assay

Examples are based on well-established in-vitro challenge model (infectious encounter with primary human immune cells) as described above. In order to characterize the ‘inflammatory signatures’ driven by SARS-CoV2, multiplex protein analyzes (qualitative and quantitative) and the molecular patterns were analyzed using multi-omics applications.

Peripheral blood mononuclear cells (PBMC) were used as a surrogate system for the sensor functions of the bodies innate immune system against the infectious agent SARS-CoV2 and the first-line-of-defense immunological activity profile is monitored using the harvested cell culture supernatants and frozen cells, respectively. To this end, a well-established assay system was further developed by using commercially available SARS-CoV2 antigens (spike protein and nucleocapsid protein) and the PBMC from SARS-CoV2 naive donors were incubated with these surrogate infectious challenges. The cell culture supernatants were qualitatively and quantitatively examined for inflammatory and regulatory proteins, mainly cytokines. Furthermore, this cell culture approach was expanded in order to test immunomodulation with approved therapeutic agents, which previously had been defined using a digital algorithm as substance screen for anti-inflammatory agents.

Interleukin 6 was defined as one of the most relevant read-out parameters, since the COVID-19-associated inflammation is also monitored in sick patients using IL-6. In addition, further inflammatory and anti-inflammatory/regulatory factors were measured in few selected samples, in order to define additional parameters possibly relevant during immunomodulation for ongoing larger experiments. These extended inflammatory/regulatory signatures shall be used for eventual future clinical studies.

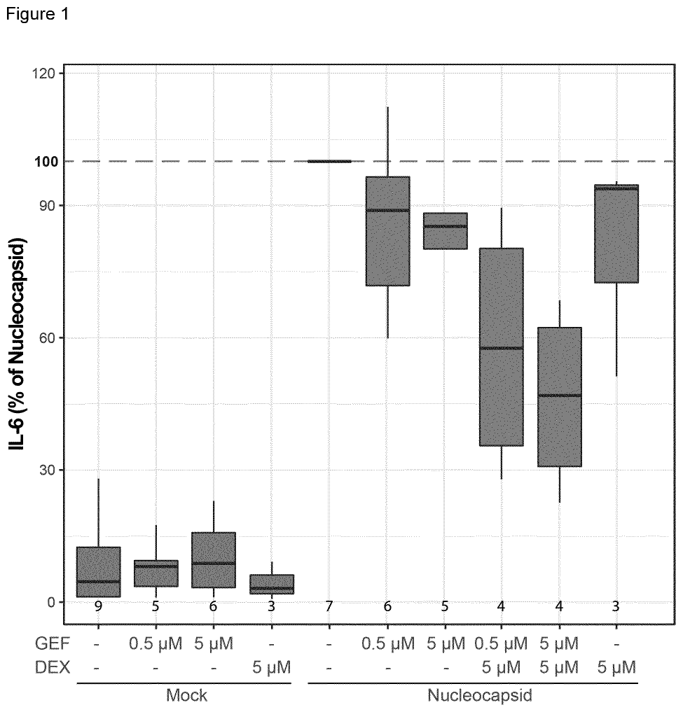

FIG. 1 shows release of interleukin 6 (IL-6) in response to nucleocapsid (N) stimulation or Mock stimulation as control. Immune modulation by gefitinib (GEF) alone was stronger than by dexamethasone (DEX) alone. The strongest immunomodulatory effect was observed in combination of GEF and DEX.

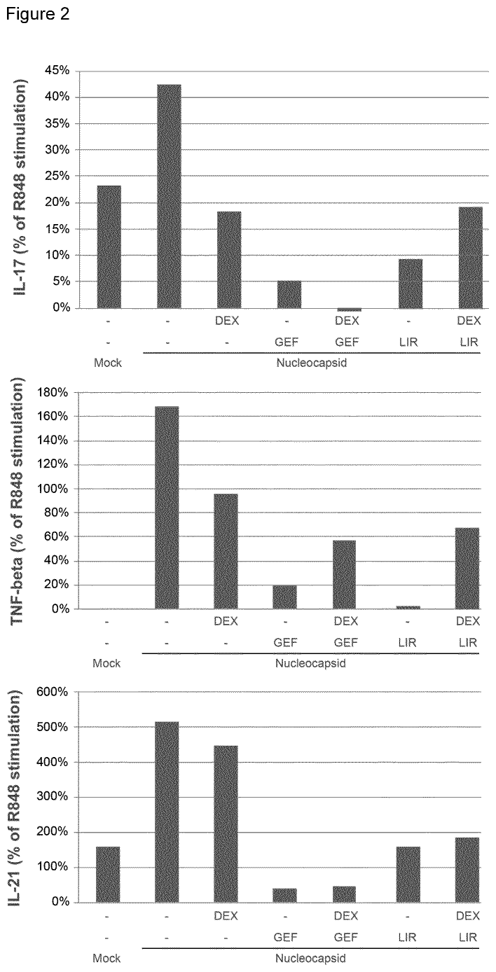

FIG. 2 shows release of further inflammatory and anti-inflammatory/regulatory factors, such as interleukin 17 (IL-17), tumor necrosis factor beta (TNF-beta) and IL-21 in response to nucleocapsid (N) stimulation or Mock stimulation as control. Immune modulation by gefitinib (GEF, 5 μM), liraglutide (LIR, 2 μg/ml), and dexamethasone (DEX, 5 μM) alone as well as in combination is shown relative to stimulation with the positive control resiquimod (R848, 200 ng/ml).

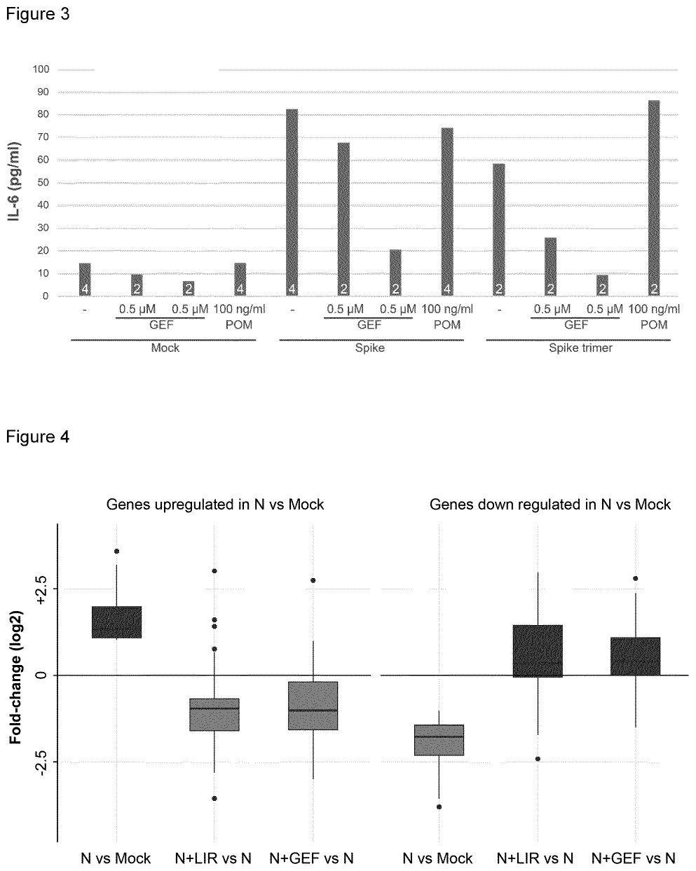

FIG. 3 shows release of interleukin 6 (IL-6) in response to spike (S), spike trimer (St) or Mock stimulation as control. Immune modulation by gefitinib (GEF, 0.5 μM and 5 μM) alone is shown in comparison to pomalidomide (POM, 100 ng/ml) as an example of a compound that was not identified with the digital substance screen, and which does not show immune modulation in this assay.

### Example 2

Transcription Fingerprinting by 3′ RNA-Sequencing

NGS Library Preparation (Lexogen QuantSeq 3′mRNA-Seq)

The amount of total RNA was quantified using the Qubit 4.0 Fluorometric Quantitation system (Thermo Fisher Scientific, Waltham, Mass., USA) and the RNA integrity number (RIN) was determined using the 2100 Bioanalyzer instrument (Agilent, Santa Clara, Calif., USA). RNA-seq libraries were prepared with the QuantSeq 3′ mRNA-Seq Library Prep Kit (FWD) for Illumina (Lexogen, Vienna, Austria). Library concentrations were quantified with the Qubit 4.0 Fluorometric Quantitation system (Life Technologies, Carlsbad, Calif., USA) and the size distribution was assessed using the 2100 Bioanalyzer instrument (Agilent, Santa Clara, Calif., USA). For sequencing, samples were diluted and pooled into NGS libraries in equimolar amounts.

Next-Generation Sequencing and Raw Data Acquisition

Expression profiling libraries were sequenced on HiSeq 3000/4000 instruments (Illumina, San Diego, Calif., USA) following a 50-base-pair, single-end recipe. Raw data acquisition (HiSeq Control Software, version 3.4.0.38) and base calling (Real-Time Analysis Software, version 2.7.7) was performed on-instrument, while the subsequent raw data processing off the instruments involved two custom programs. In a first step, base calls were converted into lane-specific, multiplexed, unaligned BAM files suitable for long-term archival. In a second step, archive BAM files were demultiplexed into sample-specific, unaligned BAM files.

Transcriptome Analysis

NGS reads were mapped to the Genome Reference Consortium GRCh38 assembly via “Spliced Transcripts Alignment to a Reference” (STAR) utilising the “basic” Ensembl transcript annotation from version e100 (April 2020, Dobin et al., Bioinformatics, 2013, 29(1), 15-21) as reference transcriptome. STAR was run with options recommended by the ENCODE project. Aligned NGS reads overlapping Ensembl transcript features were counted with the Bioconductor (version 3.12) GenomicAlignments (version 1.26.0) package via the summarizeOverlaps function in Union mode, taking into account that the Quant-seq protocol leads to sequencing of the second strand so that all reads needed inverting before counting. Transcript-level counts were aggregated to gene-level counts and the Bioconductor DESeq2 (1.30.0, Love M I, et al (2014), Genome Biology, 15, 550.) package was used to test for differential expression based on a model using the negative binomial distribution.

Results

FIG. 4 shows that those genes from the COVID-19 model that were up- or down-regulated 2-fold in the comparison of SARS-CoV2 nucleocapsid (i.e. upon stimulation with nucleocapsid (N) protein) vs Mock are “normalized” in their expression by both GEF and LIR.

For this purpose, the genes that were at least 2-fold up- or 2-fold down-regulated in the comparison of N vs Mock were extracted and compared to N+GEF vs N as well as N+LIR vs N for the same genes, respectively.

The boxplots show that the regulation was clearly reversed in its direction, e.g. the up-regulated genes at time 4 h N vs Mock were strongly down-regulated in the comparisons N-LIR vs N and N-GEF vs N).

Many of the genes represented in the proprietary molecular disease pathophysiology model are in fact reversed in their regulation compared to stimulation with SARS-CoV2 nucleocapsid. Genes that were upregulated in the comparison of nucleocapsid (N) stimulation vs Mock were downregulated through the effect of gefitinib (GEF) and/or liraglutide (LIR) or combinations with dexamethasone (DEX). Genes that were downregulated in the comparison of nucleocapsid (N) stimulation vs Mock were upregulated by GEF and/or LIR and/or or combinations with DEX.

1. A method for prophylactic or therapeutic treatment of a disease condition which is caused by or associated with an infection by a coronavirus, comprising the step of administering a pharmaceutical preparation comprising an effective amount of liraglutide, gefitinib, or a salt or solvate thereof to a subject.

2. The method of claim 1, wherein the coronavirus is a β-coronavirus.

3. The method of claim 1, wherein the pharmaceutical preparation is a medicinal product or a drug product comprising liraglutide or gefitinib and a pharmaceutically acceptable carrier.

4. The method of claim 1, wherein the disease condition is common cold, infection of the nose, throat and larynx, sinusitis, bronchiolitis, diarrhea, rash on skin, pneumonia, acute respiratory distress syndrome (ARDS), symptoms of the central nervous system, hepatic steatosis, portal fibrosis, occurrence of lymphocytic infiltrates and ductular proliferation, lobular cholestasis, acute liver cell necrosis, central vein thrombosis, renal proximal tubular injury, focal pancreatitis, adrenocortical hyperplasia, lymphocyte depletion of spleen and lymph nodes, alveolar damage specifically characterized by edema, hyaline membranes, proliferation of pneumocytes and fibroblasts, or endothelial damage.

5. The method of claim 1, wherein the antiviral effective amount is effective in preventing infection of susceptible cells by the virus, thereby treating the disease condition.

6. The method of claim 1, wherein liraglutide comprises the sequence His-Ala-Glu-Gly-Thr-Phe-Thr-Ser-Asp-Val-Ser-Ser-Tyr-Leu-Glu-Gly-Gln-Ala-Ala-Lys(1)-Glu-Phe-Ile-Ala-Trp-Leu-Val-Arg-Gly-Arg-Gly (SEQ ID NO: 1).

7. The method of claim 6, wherein the pharmaceutical preparation comprises about 50 ng to 1 g of liraglutide.

8. The method of claim 1 wherein gefitinib comprises the following structure:

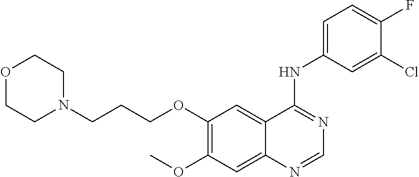 [SMILES: COc1cc2ncnc(Nc3ccc(F)c(Cl)c3)c2cc1OCCCN1CCOCC1]

9. The method of claim 8, wherein the pharmaceutical preparation comprises about 50 ng to 1 g of gefitinib.

10. The method of claim 1, wherein said pharmaceutical preparation is formulated for local administration or for systemic administration.

11. The method of claim 1, wherein said pharmaceutical preparation is administered to the subject as a spray, a powder, a gel, an ointment, a cream, a foam, a liquid solution, a lotion, a patch, a gargle solution, an aerosolized powder, an aerosolized liquid formulation, granules, or capsules.

12. The method of claim 1, wherein the pharmaceutical preparation is administered in combination with another active agent selected from the group consisting of an antiviral agent, an anti-inflammatory agent, and an antibiotic.

13. The method of claim 1, wherein the pharmaceutical preparation is administered in combination with an active agent selected from the group consisting of suramin, raloxifene, maraviroc, miglustat, quinacrine, glatiramer acetate, auranofin, and dexamethasone.

14. The method of claim 1, wherein the subject has been infected or is at risk of being infected with coronavirus.

15. A pharmaceutical preparation comprising liraglutide, gefitinib, or a salt or solvate thereof in an effective amount for prophylactic or therapeutic treatment of a disease condition which is caused by or associated with an infection by a coronavirus, further comprising an active agent selected from the group consisting of suramin, raloxifene, maraviroc, miglustat, quinacrine, glatiramer acetate, auranofin, and dexamethasone.

16. The method of claim 2, wherein the β-coronavirus is selected from the group consisting of SARS-CoV-2, MERS-CoV, SARS-CoV-1, HCoV-OC43, and HCoV-HKU1, or mutants thereof.

17. The method of claim 10, wherein said pharmaceutical preparation is formulated for nasal, pulmonary, intraoral, ocular, or dermal use or for application to the upper and lower respiratory tract.

18. The method of claim 1, wherein said pharmaceutical preparation is administered to the subject by parenteral administration.

19. The method of claim 12, wherein the pharmaceutical preparation comprises liraglutide, gefitinib, or a salt or solvate thereof as the sole active agent.


SEQUENCE LISTING (1 sequence)

SEQ ID NO: 1 - protein, 31 aa, Artificial Sequence
  liraglutide sequence
HAEGTFTSDVSSYLEGQAAKEFIAWLVRGRG